(19) United States
(12) Patent Application Publication
(10) Pub. No.: US 2013/0259801 A1
(43) Pub. Date: Oct. 3, 2013

(54) SERUM AMYLOID P PROTEIN

(71) Applicants: Catherine Chenou, Paris (FR); Jean-François Prost, Versailles (FR)

(72) Inventors: Catherine Chenou, Paris (FR); Jean-François Prost, Versailles (FR)

(73) Assignee: LABORATOIRE FRANCAIS DU FRACTIONNEMENT ET DES BIOTECHNOLOGIES, Les Ulis (FR)

(21) Appl. No.: 13/993,292

(22) PCT Filed: Dec. 16, 2011
(86) PCT No.: PCT/EP2011/073127

§ 371 Date: Jun. 11, 2013

(30) Foreign Application Priority Data
Dec. 17, 2010 (FR) 1060769

(51) Int. Cl. C07K 14/47

(52) U.S. Cl. CPC C07K 14/47
USPC 424/1.41; 530/380; 435/7.92; 530/389.3

(57) ABSTRACT

A method for purifying serum amyloid P protein from fresh unfrozen plasma, includes recalcifying the fresh unfrozen plasma and separation by phenyl-type hydrophobic interaction chromatography. The serum amyloid P protein is also obtainable by the method and the use thereof for therapeutic treatment purposes. A method for determining amyloid deposits in a tissue or organ of a subject is also provided.

### CROSS-REFERENCE TO RELATED APPLICATIONS

This application is a National Phase Entry of International Application No. PCT/EP2011/073127, filed on Dec. 16, 2011, which claims priority to French Patent Application Serial No. 1060769, filed on Dec. 17, 2010, both of which are incorporated by reference herein.

### FIELD OF THE INVENTION

The present invention concerns a method for isolating and purifying serum amyloid P protein, the human serum in particular. The invention also concerns a serum amyloid P protein obtainable by this method and the use thereof in the diagnosis or treatment and/or prevention of amyloidosis including Alzheimer's disease, or the use thereof in the therapeutic treatment of type II diabetes or autoimmune diseases in a subject. The present invention further concerns an antibody directed against serum amyloid P protein obtainable by the method of the invention. The present invention also relates to a kit for scintigraphic determination of the presence of amyloid deposits in a subject's tissue or body organ, comprising serum amyloid P protein obtainable by the method of the invention as reagent.

### BACKGROUND

Amyloidosis is a heterogeneous group of disorders due to extracellular deposits of proteins in insoluble, abnormal fibrillar forms affecting one or more body organs. Their accumulation and persistence are the cause of the perturbed structure and function of the affected tissues and leads to a serious, generally fatal illness. Although a microscopic amyloid deposit is always present in elderly persons and rarely causes clinical problems, more substantial amyloidosis in particular in the vital organs is generally associated with a progressive, non-treatable and usually fatal illness. The most frequent disorders associated with amyloidosis are Alzheimer's disease and type II diabetes, although other rarer hereditary and acquired forms of amyloidosis do exist.

The symptoms vary considerably in relation to the affected body organ. In secondary amyloidosis, treatment of the cause can delay the progression of the disease. If amyloidosis is localised, ablation of local deposits can be offered. Depending on the type of amyloidosis, different treatments can be provided such as anti-inflammatories, corticosteroids or immunosuppressants.

The diagnosis of amyloidosis can be determined after biopsy puncture and histological examination under Congo red staining, or by scintigraphy with radio-labelled iodine. For scintigraphy, the method of use is the following: the purified amyloid P component labelled with radioactive iodine is injected intravenously. This protein is then fixed by the amyloid deposits existing in the body. Examination using a gamma camera then allows visualisation of the amyloid deposits which have captured this labelled protein. On the other hand, in a healthy person, this labelled protein circulates in the blood but is not deposited on any tissue. To confirm diagnosis, a biopsy is necessary.

The scintigraphy technique also allows quantification of the mass of amyloid tissue in the body. The larger the amount of amyloid tissue, the greater the amount of labelled protein that is retained and hence its absence in 24-hour urines. It is then possible to calculate the dose of eliminated radioactive dose. The smaller this dose, the greater the body retention and hence the greater the mass of amyloid tissue.

Human serum amyloid P component is the universal constituent of amyloid deposits in all forms of amyloidosis, including cerebral amyloid deposits in Alzheimer's disease, on account of its specific calcium-dependent binding affinity with amyloid fibrils. The physiological role of human tissue amyloid P component in the pathogenesis or persistence of amyloidosis is still ill-known. It could act in vivo as co-factor in the formation of amyloid fibrils and may be involved in their protection against proteolytic degradation. On the other hand, the human tissue amyloid P component in vitro does not appear to be necessary for amyloid fibrillogenesis, this being generated from suitable precursors. In addition, the human tissue amyloid P component involved in amyloidosis is identical to its serum form. This is rapidly absorbed and catabolised in the hepatocytes, contrary to the tissue form of amyloid P component which is not catabolised in the deposits, since it is highly resistant to proteolytic degradation.

Human serum amyloid P component is a decameric plasma glycoprotein composed of identical sub-units combined in non-covalent manner in two pentamer rings interacting face to face. Each sub-unit has a molecular weight of 25 kDa and theoretical pHi of 6.1. Human serum amyloid P component is naturally present in the plasma at a level generally of between 30 and 40 μg/mL. In the plasma, human serum amyloid P component is the major binding protein to DNA, to chromatin and other ligands such as fibronectin, the C4-binding protein, glycosaminoglycans, phosphocholine, the C1q molecule (collagen-like C1q molecule).

Current techniques for purifying serum amyloid P component allow a purified human serum amyloid P protein (SAP) to be obtained that can be used after labelling with a radio-isotope in scintigraphy for the diagnosis of amyloidosis. Nonetheless, these purifying methods can still be improved both regarding method and regarding biological safety. There is a real need for methods which allow a high yield but which also lead to increased stability and purity. Optimisation of biological safety is also one of the current major challenges to ensure the removal of any contaminant, pathogenic agent, virus or prion and to guarantee viral safety conforming to regulations in force. Human serum amyloid P component is described as a calcium-dependent binding protein having high affinity for agarose.

De Beer et al, 1982 (De Beer F C, Pepys M B, “Isolation of human C-reactive protein and serum amyloid P component” J. Immunol. Methods 1982; 50(1):17-31) teach a method for isolating and purifying human serum amyloid P component comprising successive steps of calcium-dependent affinity chromatography on non-substituted Sepharose 4B (agarose) followed by reverse affinity chromatographies specifically binding some contaminants, but not having affinity for the serum amyloid P component (Sepharose-anti-normal human serum and optionally Blue-Sepharose) and gel filtration (Sephacryl S-300). Elution is performed in Tris-saline-EDTA solution. Their yield after gel filtration is about 14.7 μg/mL of serum with an indicated purity of 100%. However, having regard to the reproducibility difficulties related to the quality of the agarose, it appears that the use of this type of chromatography is not indicated for purifying human serum amyloid P protein on an industrial scale.

Urbanyi Z. et al, 1992 (Urbanyi Z., Medzihradszky D. “Rapid method to isolate serum amyloid P component from human plasma. Characterization of the isolated protein” J. Chromatogr. 1992; 578(1):130-3) teach a protocol for isolating and purifying human serum amyloid P component comprising a recalcification step, a separation step by affinity chromatography on Sepharose 6B (agarose) with elution of the human serum amyloid P protein in Tris saline EDTA solution, pH7.8, and an isolation step by ion exchange chromatography with elution using a 0.3 M salt solution. Their yield is 8.5 μg/mL of serum. This publication in 1992 discloses a recalcification step of frozen human plasma in the presence of citrate salts, followed by centrifugations; on the other hand no other chromatographic process is mentioned. The first separation step by calcium-dependent affinity chromatography on agarose, according to Urbanyi Z. et al, 1992, allows the separation of serum amyloid P protein from most other plasma proteins, and the separation step by hydrophobic interaction chromatography, according to Urbanyi et al, allows a purified protein to be obtained without specifying the degree of purity however. In addition, Urbanyi Z. et al, 1992 do not disclose any virus inactivation step.

Mantovaara T. et al, 1989 (Mantovaara T. et al, “Purification of human serum amyloid P component (SAP) by calcium affinity chromatography” Biotechnology and Applied Biochemistry, 1989; 11:564-570) teach a single-step method for purifying human serum amyloid P component by calcium-dependent affinity chromatography on non-substituted agarose beads coupled to carboxy-methylated aspartic acid (Ca-CM-Asp-agarose). The human serum amyloid P protein is adsorbed on the agarose column Ca-CM-Asp-agarose in the presence of 0.25 M CaCl₂ and is eluted at pH5.0 in the presence of EDTA. The indicated yield is 36 μg/mL of serum which corresponds to 90% recovery of SAP protein, their purification factor is in the order of 1900. However, Mantovaara T. et al, 1989 mention that elution of human serum amyloid P protein in the presence of EDTA may modify the conformation and even the biological properties of this protein; therefore the use of EDTA in the eluting solution does not appear to be a recommended approach. In addition this purification mode reports heavy contamination with immunoglobulins: therefore despite a high yield this purification method can still nevertheless be improved. The addition of CaCl₂ is simply indicated for calcium-dependent affinity chromatography on non-substituted agarose beads coupled to carboxy-methylated aspartic acid (Ca-CM-Asp-agarose).

Hawkins P N et al, 1991 (Hawkins P N et al, “Studies in vivo and in vitro of serum amyloid P component in normals and in a patient with AA amyloidosis” Clin. Exp. Immunol. 1991; 84:308-316) teach a protocol for isolating and purifying serum amyloid P component comprising the following successive steps: (i) calcium-dependent chromatography on phosphoethanolamine-Sepharose, with elution in Tris saline EDTA buffer in the presence of the antibacterial agent NaN₃, pH8.0; (ii) successive chromatographies on protein A-Sepharose, Blue-Sepharose, then anti-impurity-Sepharose column (immunisation of sheep with serum depleted of serum amyloid P protein, recovery of the immunoglobulin G fraction of anti-serum and coupling of this fraction on CNBr-Sepharose); (iii) concentration by ultrafiltration and dialysis (iv); calcium-dependent chromatography on phosphoethanolamine-Sepharose column eluting with a Tris saline NaN₃ solution, pH8.0. With this method, Hawkins P N et al, 1991 obtain a mean concentration of human serum amyloid P protein of 37 μg/mL of plasma, irrespective of the genotype under consideration, and whether or not the donors are healthy persons or patients suffering from amyloidosis or suffering from a disease predisposing to the development of amyloidosis.

Patent application WO 95/14234 relates to a diagnosis test for detecting Alzheimer's disease, and for monitoring the progression of this disease, by measuring the level of amyloid P protein in a sample of cerebrospinal fluid of a given patient. The measured plasma concentration of human serum amyloid P protein generally averages 31 mg/mL in healthy individuals. However, no method for purifying human serum amyloid P protein is described.

### SUMMARY

These requirements have allowed the development of a novel method for purifying human serum amyloid P component, comprising the steps of recalcifying fresh unfrozen plasma, hydrophobic interaction chromatography, preferably pseudo-affinity chromatography on hydroxyapatite, and more preferably affinity chromatography on protein A. Aside from the considerable improvement in yield (12 to 17 mg/L of plasma i.e. 34.8 to 48.6%), this method allows the industrial obtaining of a concentrate of serum amyloid P protein SAP having a high degree of purity (notably 98.6%) that is biologically safe and therefore able to be used for diagnostic purposes, for monitoring treatment and/or the prevention of amyloidosis including Alzheimer's disease, or in the therapeutic treatment of type II diabetes or autoimmune diseases in a subject. This method also allows the preserving of the biological properties of serum amyloid P protein obtainable by the method of the invention, and more particularly its binding capacity with natural ligands of serum amyloid P protein such as phosphoethanolamine, fibronectin, the C1q molecule, collagen, la phosphocholine, DNA and chromatin, immune complexes, sugars and in particular methyl 4,6-O-(1-carboxyethylidene)-β-D-galactopyranoside (MOβDG), complement components, all forms of amyloid fibrils, C4-binding protein and glycosaminoglycans.

The method of the invention sets itself apart most particularly from purification methods known to date through the use of hydrophobic groups particularly of phenyl type for the chromatography of a recalcified supernatant of plasma cryoprecipitate. With said method, it is possible to apply a solvent/detergent treatment step on the eluate derived from this hydrophobic interaction chromatography, and in addition the flow derived from the solvent/detergent treatment can be treated directly at a subsequent pseudo-affinity chromatography step on hydroxyapatite and, if need, can be subject to an affinity chromatography step on protein A before being nanofiltered.

The Applicant has surprisingly found that a method for purifying human serum amyloid P component, combining the steps of recalcifying fresh unfrozen plasma, hydrophobic interaction chromatography, preferably pseudo-affinity chromatography on hydroxyapatite and optionally affinity chromatography on protein A, allows the industrial preparation of a concentrate of human serum amyloid P component having a high degree of purity. The degree of purity attained when implementing the method of the invention, after separation step (c) by pseudo-affinity chromatography on hydroxyapatite is 94% or higher, preferably 95% or higher, more preferably 96% or higher, further preferably 97% or higher, and most advantageously higher than 98%. In particular the degree of purity achieved when implementing the method of the invention, after separation step (d) by affinity chromatography on protein A, is 96% or higher, preferably 97% or higher, more preferably 98% or higher, and further preferably 99% or higher. This concentrate prepared according to the invention is substantially free of contaminant proteins and in particular is free of immunoglobulins, preferably immunoglobulins M and G, and of C3 complement factors, this list not being limiting. The method of the invention differs most particularly from purification methods known to date through the use of hydrophobic groups in particular of phenyl type for the chromatography of a recalcified supernatant of plasma cryoprecipitate containing proteins of high molecular weight such as fibrinogen, fibronectin, immunoglobulins, complement proteins, this list not being limiting. With said method, it is possible to apply a solvent/detergent treatment step on the eluate derived from this hydrophobic interaction chromatography, and in addition the flow derived from the solvent/detergent treatment can be directly treated at a subsequent pseudo-affinity chromatography step on hydroxyapatite, followed if necessary by subjection to an affinity chromatography step on protein A before being nanofiltered. With said method, it is also possible to obtain a purified serum amyloid P protein that is virally safe and is therefore able to be used for diagnostic purposes, for the monitoring of treatment and/or prevention of amyloidosis including Alzheimer's disease, or for use in the therapeutic treatment of type II diabetes or autoimmune diseases in a subject.

Compared with the method described in the De Beer publication, the introducing of the steps of recalcifying fresh unfrozen plasma, hydrophobic interaction chromatography, pseudo-affinity chromatography on hydroxyapatite and affinity chromatography on protein A in the novel method for purifying human serum amyloid P component, allows a production mode on industrial scale to be obtained on account of the reproducibility of these steps. In addition, in the method developed by De Beer, there is no mention of any process to obtain bacterial or viral safety, the solvent/detergent step therefore brings real progress in terms of virus inactivation. Compared with the publication by Urbanyi Z., it will be noted that the physicochemical parameters on which the separation principles are based relating to affinity chromatographies on Sepharose 6B (agarose), are fully different from those used in ion exchange chromatography, hydrophobic interaction chromatography and pseudo-affinity chromatography on hydroxyapatite.

The invention therefore provides a method for purifying serum amyloid P protein from a starting material characterized in that the said method comprises the following steps:

a) a recalcification step;

b) a separation step by hydrophobic interaction chromatography.

A further subject of the invention is the serum amyloid P protein obtainable by the method of the invention. This protein has a binding capacity with a natural ligand of the serum amyloid P protein which is preserved on completion of the method of the invention. This protein binds at the binding site of amyloid fibrils and additionally has strong affinity for amyloid fibrils.

A further subject of the invention is an antibody directed against the serum amyloid P protein obtained with the method of the invention. A further subject of the invention is a method for determining the presence of amyloid deposits in a tissue or body organ of a subject, characterized in that the method comprises the following steps: (i) systemic administering of the amyloid P protein of the invention; (ii) using a gamma camera, detection of a signal in the tissue or organ, said signal being emitted by the protein; and (iii) obtaining at least one image of the tissue or organ, the image being obtained as output data from a gamma-camera. A further subject of the invention is a method for determining the presence of amyloid deposits in a tissue or organ of a subject (diagnostic screening test) characterized in that the method comprises the following steps: (i) systemic administering of the amyloid P protein according to the invention; (ii) quantification in a biological sample of the subject of the signal emitted by the administered protein; and (iii) determining a ratio between the quantity of protein quantified at step (ii) and the quantity of protein administered as step (i).

The method for determining the presence of amyloid deposits in a subject's tissue or organ according to the present patent application therefore differs from patent application WO 95/14234 in that the quantification of the signal emitted by the P protein, purified according to the novel purification method and administered systemically, is determined either by scintigraphy by means of a gamma-camera, or by sampling a biological fluid of said subject, preferably blood or urine, and by determining a ratio between the quantity of protein quantified in the sample and the quantity of administered protein. A further subject of the invention is a kit for determining by scintigraphy the presence of amyloid deposits in a tissue or organ of a subject, which as reagent comprises the serum amyloid P protein obtained with the method of the invention and radiolabelled.

A further subject of the invention is the amyloid P protein of the invention for the diagnosis or therapeutic and/or prophylactic treatment of amyloidosis including Alzheimer's disease in a subject, or for the diagnosis or treatment of type II diabetes in a human, or for the diagnosis or treatment of autoimmune diseases in a subject. The amyloid P protein of the invention is used in the treatments of the invention as ligand or vector to convey a therapeutic molecule of interest.

### DRAWINGS

FIG. 1: Schematic summarising the purification steps of human serum amyloid P protein according to one particular embodiment of the invention.

FIG. 2: Coomassie Blue Gel (Blue Safestain, Invitrogen) of the eluate leaving the hydrophobic interaction chromatography column-Gel Novex Tris glycine 4-20% (InVitrogen, EC6025 Box), 40 mA constant in Tris-Glycine-SDS (TGS) 1× buffer containing 0.025 M of base Tris, 0.192 M of Glycine, 0.1% (v/v) of SDS in 1 L of water at pH 8.3±0.2-Denaturation of the samples at +95° C. for 5 minutes in standard denaturing buffer SB 5×, containing 0.313 M Tris-HCl, 50% glycerol (v/v), 100 g/L SDS, 0.5 g/L BBP and 25% (v/v) of 2-mercaptoethanol in a solution at pH 6.8.-MW well: Molecular weight marker (BenchMarkproteinladder, InVitrogen, 10747-012), 5 μl-Well 1: Loading-Well 2: Non-retained fraction/Washing-Well 3: Washing 1-Well 4: Washing 2-Well 5: Washing 3-Well 6: Elution peak-Well 7: End Elution-Well 8: Regeneration-Well 9: Elution peak (assay 1 μg).

FIG. 3: Coomassie Blue Gel (Simply Blue Safestain, Invitrogen) of the eluate leaving the chromatography on hydroxyapatite column-Gel Novex Tris glycine 4-20% (InVitrogen, EC6025 Box), 40 mA constant in TGS 1× buffer-Denaturation of samples at +95° C. for 5 minutes in standard denaturing buffer SB 5×. MW well: Molecular weight marker (BenchMarkproteinladder, InVitrogen, 10747-012), 5 μl-Well 1: Loading-Well 2: Non-retained fraction/Washing-Well 3: Washing 10 mM PO4-Well 4: Washing 60 mM PO4-Well 5: Elution 100 mM PO4-Well 6: Regeneration-Well 7: End Elution-Well 8: Elution 100 mM PO4 (10 μg).

FIG. 4: Western-blot gel-Molecular weight marker (BenchMarkPre-stainedproteinladder, InVitrogen, 10748-010): 10 μl-Primary Ab: Rabbit anti-Serum Amyloid P Component (Calbiochem, 565191), 1/7500, 1H-Secondary Ab: Anti-rabbit Alkaline Phosphatase conjugated (Promega, S373B), 1/7500, 1H-Detection: NBT/BCIP-Well 1: Molecular weight marker (5 μL)-Well 2: Sample Buffer 1× (20 μL)-Well 3: R125/Pool Hydroxyapatite concentrate (10 μg)-Well 4: Sample Buffer 1× (20 μL)-Well 5: 0.2% BSA (0.02 μg)-Well 6: 0.5% BSA (0.05 μg)-Well 7: 1% BSA (0.10 μg)-Well 8: 1.5% BSA (0.15 μg)-Well 9: 2% BSA (0.20 μg)-Well 10: 4% BSA (0.40 μg).

FIG. 5: Competitive binding assay whose results are given in graph form with optical density value (OD) along the X-axis measured at 450-620 nm for increasing concentrations of free fibronectin (μg/ml) FIG. 5A: fibronectin-coated substrate 10 μg/ml; serum amyloid P protein obtained with the method of the invention, or commercially available serum amyloid P protein (standard) 12.5 μg/ml.

FIG. 5B: fibronectin-coated substrate 10 μg/ml; serum amyloid P protein obtained with the invention or standard 12.5 μg/ml-FIG. 5C: fibronectin-coated substrate 10 μg/ml; serum amyloid P protein obtained with the method of the invention or standard 50 μg/ml.

FIG. 6: SDS-PAGE Gel under reducing or non-reducing conditions before affinity chromatography on protein A.

FIG. 7: Coomassie Blue Gel (Simply Blue Safestain, Invitrogen) of the eluate leaving the affinity chromatography column on protein A-Novex Tris glycine Gel 4-20% (InVitrogen, EC6025 Box), 40 mA constant in Tris-Glycine-SDS buffer (TGS) 1× containing 0.025M base Tris, 0.192M Glycine, 0.1% (v/v) SDS in 1 L of water at pH 8.3±0.2-Denaturing of the samples at +95° C. for 5 minutes in standard denaturing buffer SB 5×, containing 0.313 M Tris-HCl, 50% glycerol (v/v), 100 g/L SDS, 0.5 g/L BBP and 25% (v/v) of 2-mercaptoethanol in a solution at pH 6.8-MW well: Molecular weight marker (BenchMarkproteinladder, InVitrogen, 10747-012), 1 μl-Well 1: Loading-Well 2: Non-retained fraction/Washing-Well 3: regeneration.

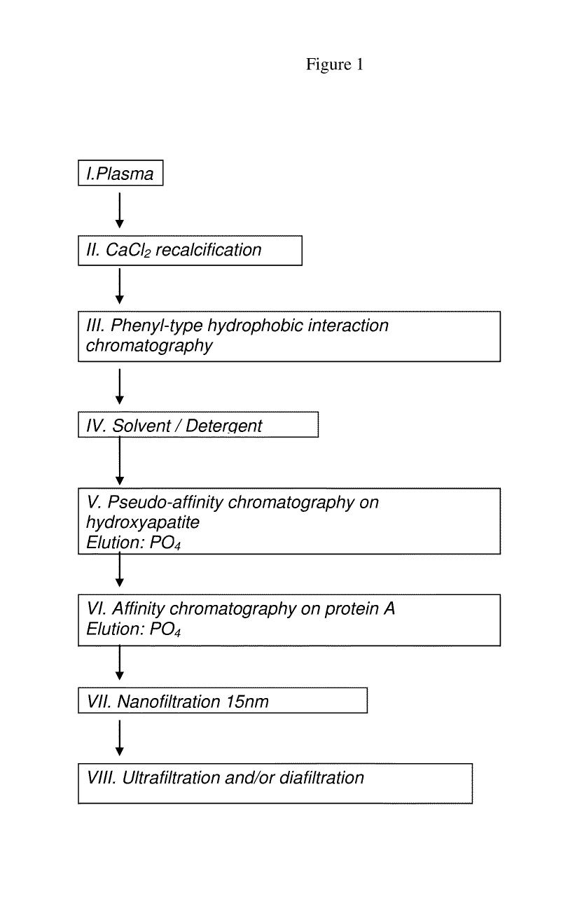

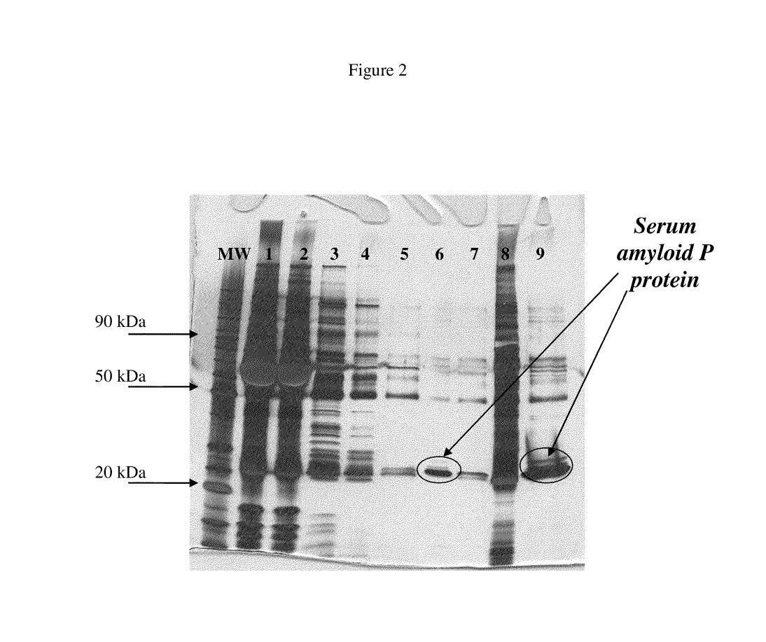

### DETAILED DESCRIPTION

In the present invention by “amyloidosis” or “amyloid disease” is meant a disease characterized by the extracellular accumulation of tissue amyloid P component in various organs and tissues of the body. Amyloidosis, which is a disease directly cause by amyloid deposit in the tissues, comprises both local amyloidosis in which the deposits are limited to one anatomic region and/or to a tissue or organ, and systemic amyloidosis in which the deposits may occur in any type of body organ or tissue.

By “serum amyloid protein” is designated any form of serum amyloid P protein having calcium-dependent affinity for deposits of amyloid fibrils. This term also encompasses biologically active derivatives of serum amyloid P protein such as those for example having undergone one or more post-translational modifications, or any other form naturally present in the blood. By “serum amyloid protein” is also meant the tissue amyloid P component found in amyloid deposits and which is identical to the serum amyloid P protein.

By “natural ligand of serum amyloid protein” is meant any natural substance or derived from a natural substance capable of binding with serum amyloid P protein in calcium-dependent manner such as phosphoethanolamine, fibronectin, the C1q molecule, collagen, phosphocholine, DNA and chromatin, immune complexes, sugars and in particular methyl 4,6-O-(1-carboxyethylidene)-β-D-galactopyranoside (MOβDG), complement components, all forms of amyloid fibrils, the C4-binding protein and glycosaminoglycans.

By “antibody directed against the serum amyloid P protein obtainable by the method of the invention” is meant an immunoglobulin molecule or derivative fragment thereof capable of interacting and of specifically binding with an epitope of serum amyloid P protein or with a derivative fragment thereof.

By “diagnosis of amyloidosis” is meant the identification of all forms of amyloidosis, whether localised or systemic. The most frequent forms are primary and secondary AL amyloidosis (immunoglobulin disorders), AA amyloidosis (inflammatory) secondary to chronic inflammatory diseases (rheumatoid polyarthritis . . . ), hereditary ATTR amyloidosis (transthyretin), dialysis-related amyloidosis, familial Mediterranean fever amyloidosis, Alzheimer's disease.

By “autoimmune diseases” is meant a disease characterized by rupture of tolerance mechanisms which leads to pathogenic action of the immunity system against the natural constituents of the body. Among autoimmune diseases, a distinction is made between organ-specific autoimmune diseases such as type I diabetes, autoimmune thyroiditis, autoimmune hepatopathy, myasthenia, autoimmune blistering diseases, vitiligo, autoimmune uveitis, autoimmune retinitis, autoimmune cytopenia, systemic autoimmune diseases (non-organ specific) such as systemic lupus, Gougerot-Sjögren syndrome, rheumatoid polyarthritis, scleroderma, polymyositis and dermato-polymyositis, mixed connective tissue disease, primary vascular disease and atrophic polychondritis.

By “scintigraphy” is meant a medical imaging method which uses the systemic administering to the body of radioactive isotopes to produce a medical image by detection of the radiation emitted by these isotopes after they have been captured by the organs being examined. For this purpose, a vector molecule combined with a radioactive tracer is injected into the subject. The vector molecule locates itself selectively on a particular structure of the body. The radioactive tracer acts as “emitter” and gives information on its location. The radioactive tracer emits radiation that is both adapted to detection thereof and is of very low toxicity. The time to fixing on the target organ is variable, which accounts for the waiting time between injection and image acquisitions. Scintigraphy can relate to an imaging system comprising a gamma-camera (scintillation camera) capable of detecting and forming an image at the source of gamma radiation. The imaging system may also comprise computer equipment and software intended to produce an image in a form apparent to the observer, and to analyse the image to obtain information such as intensity of the gamma radiation emitted and the location thereof.

By “monitoring amyloidosis treatment” is meant any action (such as administering one or more compounds or pharmaceutical compositions) initiated after the onset of clinical signs of amyloidosis, so as to monitor temporarily or definitively, the progression to a clinical sign or the progression of amyloidosis.

By “prevention of amyloidosis” is meant any action (such as administering one or more compounds or pharmaceutical compositions) which is initiated before the onset of a clinical symptom of amyloidosis so as to prevent, eliminate or reduce, temporarily or permanently, the onset of a clinical sign of amyloidosis in a healthy patient or a patient suffering from amyloidosis or having a risk of amyloidosis including Alzheimer's disease. The amyloid P protein according to the invention is then bound to the therapeutic molecule.

By “therapeutic treatment of amyloidosis” is meant any action (such as administering one or more compounds or pharmaceutical compositions) initiated after the onset of a clinical symptom of amyloidosis (including Alzheimer's disease), so as to eliminate or reduce, temporarily or permanently, the onset of a clinical sign of amyloidosis. The amyloid P protein of the invention is then bound to the therapeutic molecule.

By “therapeutic treatment of type II diabetes or autoimmune disease” is meant any action (such as the administering of one or more compounds or pharmaceutical compositions) initiated after the onset of a clinical symptom of type II diabetes or autoimmune disease, so as to eliminate or reduce temporarily or permanently the onset of a clinical sign of type II diabetes or autoimmune diseases in a patient suffering from type II diabetes or autoimmune diseases. The amyloid P protein of the invention is then bound to the therapeutic molecule.

By “subject” is meant any animal, preferably mammalian, preferably of human origin.

By “purification method” is meant a method allowing the serum amyloid P protein to be separated from the other molecules contained in the starting material. These molecules may be different proteins of the serum amyloid P protein, viruses, bacteria, spores, culture medium, foetal calf serum, this list not being limiting.

By “starting material” is meant a sample containing the serum amyloid P protein. The starting material may be of recombinant, transgenic or plasmatic origin. If the starting material is of recombinant origin, it is derived from a unicellular system, in which the expression of the serum amyloid P protein has been induced by transfection of a vector containing the gene coding for serum amyloid P protein, preferably the gene encoding the human protein, in animal or human cell lines. Said cell systems express the serum amyloid P protein using techniques well known to the person skilled in the art. If the starting material is of transgenic origin, it is derived from a pluricellular system, in particular an animal or plant obtained by transgenesis, i.e. in which one or more cells have received a recombinant DNA molecule. For example, mention can be made of dogs, cats, mice, rats, hamsters, cows, goats, sheep, rabbits and swine, horses, insects, plants e.g. tobacco, soybean, this list not being limiting. With regard to animals producing the serum amyloid P protein, this production can occur in the blood or in various media secreted by the animal e.g. urine, saliva, milk, this list not being limiting. Said production methods can be performed using techniques well known to the person skilled in the art. If the starting material is of plasmatic origin, it may be plasma of animal origin, preferably mammalian and preferably human.

By “biological sample” is meant a sample containing the serum amyloid P protein and which is a biological fluid. The biological sample is notably blood, urine, saliva, extracts of these fluids such as plasma or serum.

By “plasma” is meant the liquid part of blood in which the cells are in suspension. The plasma can be separated from the cellular part of a whole blood sample for therapeutic use in the form of frozen fresh plasma or for subsequent conversion to a cryoprecipitate and to plasma free of cryoprecipitate for transfusion purposes. It can be used to manufacture medicinal products derived from human blood or human plasma, or in the preparation of mixtures of concentrates of platelets or mixtures of concentrates of leukocyte-free platelets.

By “fresh frozen plasma” is meant the supernatant plasma separated from donated whole blood or from plasma sampled by apheresis, frozen and stored. Fresh frozen plasma is stored at a temperature of −25° C. for a maximum time of one year.

By “recalcification step” is meant incubation of the starting material in a solution of calcium salts, preferably calcium chloride (CaCl₂), preferably in a buffer solution of CaCl₂ at a final concentration of between 1 mM and 50 mM, advantageously at a final concentration of between 5 mM and 20 mM, and preferably at a final concentration of 10 mM. Preferably, the recalcification step is conducted at substantially ambient temperature (e.g. 25±1° C.) for 2 hours under light agitation (100 rpm). In one particular embodiment, the recalcification step is performed by incubation of the starting material in a solution also containing magnesium salts, preferably magnesium chloride (MgCl₂), preferably in a buffer solution of MgCl₂ at a final concentration of between 1 mM and 50 mM, advantageously at a final concentration of between 5 mM and 20 mM, and preferably at a final concentration of 10 mM.

By “hydrophobic interaction chromatography” is meant a technique for separating proteins according to their hydrophobic properties. In this type of chromatography, the hydrophobic groups such as the C₆, C₈, C₁₆, C₁₈ hydrophobic groups or such as phenyl, octyl, butyl are fixed to the stationary column. The proteins which pass through the column and which have hydrophobic regions on their surfaces, are capable of interacting and binding with the hydrophobic groups fixed to the column. These interactions require the addition of a buffer solution of high ionic strength and with strong salt concentration to reduce solvation of the sample solutes and allow the adsorption of the side chains of the hydrophobic amino acids thus exposed. The more the molecule of interest is hydrophobic, the lower the necessary amount of salt content in the buffer solution for binding to occur. To elute the proteins, the salt concentration of the elution solutions is decreased to reduce hydrophobicity. Among the salts preferably used in the present invention for elution, mention can be made of Na₂SO₄, K₂SO₄, (NH₄)₂SO₄, NaCl, NH₄Cl, NaBr, NaSCN, and preferably NaCl.

By “chelating agents” is meant any agent having a particular affinity for alkaline-earth salts and metals, with which it forms stable soluble compounds, where the associated element loses its ionic properties. Examples of chelating agents capable of chelating calcium are ethylene diamine tetraacetic acid (EDTA), ethyleneglycol-bis(beta-aminoethylether)-N,N,N tetraacetic acid (EGTA) or a citrate.

By “affinity chromatography” is meant a method for purifying particular substances of a solution by making use of their capacity to set up a specific bond with a known molecule or molecules. The mixture to be purified is injected through a column containing a solid medium on which the binding molecule is covalently bound. There are several types of affinity chromatography such as immuno-affinity chromatography, pseudo-affinity chromatography or affinity chromatography for metal ions.

By “affinity chromatography on protein A or protein G” is meant chromatography having as binding molecule bound to the solid chromatography medium a protein A or a molecule derived from a protein A (affinity chromatography on protein A) or a protein G or a molecule derived from a protein G (affinity chromatography on protein G). Preferably, affinity chromatography on protein A uses an agarose matrix on which a binding molecule is fixed derived from stable protein A under alkaline conditions. Preferably, affinity chromatography on protein A is affinity chromatography of MabSelectSuRe type.

By “protein A” is meant a protein originally discovered in the cellular wall of Staphylococci which binds specifically with an Fc portion of an immunoglobulin G (IgG). For the purposes of the present invention, protein A is a protein identical or similar to protein A of Staphylococcus, which in particular is commercially available and/or recombinant forms of protein A, having the capacity to bind with an Fc portion of an IgG.

By “protein G” is meant a protein originally discovered in the cellular wall of Streptococci which binds specifically with an Fc portion of an IgG. For the purposes of the present invention, protein G is a protein identical or similar to protein G of Streptococci which in particular is commercially available and/or recombinant forms of protein G having the capacity to bind with a, Fc portion of an IgG.

By “pseudo-affinity chromatography” is designated a method for purifying particular substances of a solution combining several chromatography separation methods at the same time. Advantageously, pseudo-affinity chromatography according to the invention combines affinity chromatography with at least one other chromatography technique such as ion exchange chromatography. Preferably pseudo-affinity chromatography according to the invention is chromatography on hydroxyapatite.

By “hydroxyapatite” is designated a calcium phosphate inorganic compound having the empirical formula Ca₅ (PO₄)₃(OH). In the natural state, this compound is essentially found in bones and tooth enamel. The different binding mechanisms of hydroxyapatite are used in chromatographic separations in which it chiefly acts as adsorbent, but it also has the properties of an ion exchange material and allows the separation of proteins, enzymes, immunoglobulins and nucleic acids. Various hydroxyapatite materials are known. The hydroxyapatite resin used in the invention for example may be ceramic-Hydroxyapatite, Biogel HT, etc.

By “chromatography on hydroxyapatite” is meant chromatography on a hydroxyapatite substrate. The hydroxyapatite substrate is a mixed-mode substrate. The hydroxyapatite substrate chiefly allows two types of interactions: interactions resulting from the affinity of the protein carboxyl groups for the calcium ions of hydroxyapatite, and ionic bonds between the positively charged amino groups of a protein and the negative charges of the phosphate groups of hydroxyapatite. The carboxyl residues of a protein bind with the hydroxyapatite substrate on account of their affinity for calcium, and the amine residues bind by cation exchange. The interactions involving calcium can only be eluted with ions having strong affinity for calcium e.g. phosphate. The cation exchange interactions can be eluted with salt. Advantageously, chromatography on hydroxyapatite is chromatography on a hydroxyapatite column of ceramic hydroxyapatite type. The chromatography on hydroxyapatite according to the invention allows the removal of high molecular weight proteins which are eluted with the serum amyloid P protein at step (b) comprising hydrophobic interaction chromatography. The removal of these high molecular weight proteins contributes towards obtaining a purified serum amyloid P component. It follows that the eluate of the serum amyloid P protein resulting from chromatography on hydroxyapatite is significantly enriched.

By “solvent-detergent” is meant any suitable solvent-detergent mixture known to those skilled in the art and preferably composed of a Tween (polysorbate 80)—TnBP mixture, preferably a mixture of 1% (v/v) polysorbate 80-0.3% (v/v) TnBP mixture. Virus inactivation treatment by solvent-detergent is generally performed for a time of a few hours (e.g. 6 hours) at substantially ambient temperature (e.g. 25±1° C.). In another embodiment, the virus inactivation treatment by solvent-detergent is conducted for a time of about 16 hours at a temperature of +4° C. In one particular embodiment, the virus inactivation treatment is conducted in the form of treatment with tri-n-butylphosphate, Triton X-100 or sodium cholate.

By “diafiltration” of a solution is meant any suitable protein clarification and purification step known to skilled persons, consisting of separating proteins by filtering a solution through a membrane, preferably a Millipore Polyethersulfone membrane, via pressure difference, and adding water or a suitable solution to the retentate held back on the membrane for best removal of impurities and small molecules (sugars, salts . . . ) contained therein.

By “concentration” of a solution is meant any suitable step consisting of removing a greater or lesser portion of the water it contains, using different methods known to skilled persons. Therefore the eluate derived from this concentration step is significantly enriched with the protein of interest.

By “nanofiltration” of a solution is meant any liquid phase separation method known to skilled persons, using tangential filtration through a semi-permeable membrane, preferably a Planova 15N (Asahi) or Viresolve Pro Modus 1.1. filter, under the effect of pressure and allowing the removal of a more or less large portion of impurities, bacteria or viruses contained therein.

In one particular embodiment, the recalcification step (a) corresponds to incubation of the starting material in CaCl₂ solution, preferably a buffer CaCl₂ solution at a final concentration of between 1 mM and 50 mM, advantageously at a final concentration of between 5 mM and 20 mM, and preferably at a final concentration of 10 mM. Preferably the recalcification step is performed at substantially ambient temperature (e.g. 25±1° C.) for 2 hours and under light agitation (100 rpm). Advantageously, the starting material is fresh frozen plasma. Preferably, the fresh plasma is left to thaw for 16 to 20 hours at a temperature of between +2° C. and +8° C. In one particular embodiment, the fresh plasma is left to thaw for 16 to 20 hours preferably at ambient temperature.

Advantageously the recalcification step (a) is preceded by an incubation step of the starting material at substantially ambient temperature (e.g. 25±1° C.) for 1 hour. Advantageously, the recalcification step (a) is followed by a phase separation step, preferably by centrifuging to a supernatant. Preferably, centrifuging is performed for 20 minutes at a speed of 4000 g and temperature of +4° C. In one particular embodiment, centrifuging is performed for 20 minutes at a speed of 4000 g and preferably at ambient temperature.

In one particular embodiment, the separation step by hydrophobic interaction chromatography (b) is implemented on a stationary phase comprising hydrophobic groups of phenyl type. Advantageously, the separation step by hydrophobic interaction chromatography (b), as equilibration and/or wash solution, uses a buffer solution containing CaCl₂ at a concentration of between 1 mM and 50 mM, advantageously at a concentration of between 5 mM and 20 mM, and more preferably at a concentration of 10 mM. In one particular embodiment, the wash solution also comprises sodium salts, preferably sodium chloride (NaCl) at a concentration of between 50 mM and 1.5 M, preferably between 100 mM and 1 M, more preferably between 150 mM and 500 mM. Advantageously, the stationary column of hydrophobic interaction chromatography is washed with wash solutions having increasing concentrations of sodium salts, preferably increasing concentrations of NaCl.

In one preferred embodiment, the separation step by hydrophobic interaction chromatography (b), as equilibration and/or wash solution, uses a solution of 20 mM Tris, 10 mM CaCl₂, pH 7.5. In one particular embodiment, the stationary column of hydrophobic interaction chromatography is washed with a first solution of 20 mM Tris, 10 mM CaCl₂, pH 7.5, then with 20 mM Tris, 10 mM CaCl₂, pH 7.5 wash solutions with increasing NaCl concentration. Preferably, the stationary column of hydrophobic interaction chromatography is washed with a solution of 20 mM Tris, 10 mM CaCl₂, pH 7.5, and/or a solution of 20 mM Tris, 10 mM CaCl₂, 150 mM NaCl, pH7.5, and/or a solution of 20 mM Tris, 10 mM CaCl₂, 500 mM NaCl, pH 7.5 and/or a solution of 20 mM Tris, 10 mM CaCl₂, 1 mM NaCl, pH 7.5. Advantageously, the separation step by hydrophobic interaction chromatography (b), as eluting solution, uses a buffer solution free of CaCl₂ and/or free of chelating agent, preferably a saline buffer solution, advantageously whose salt concentration is higher than 150 mM, advantageously a salt concentration higher than 300 mM, advantageously a salt concentration higher than 500 mM, and preferably a salt concentration of 1 M, the salt preferably being NaCl.

In one preferred embodiment, the separation step hydrophobic interaction chromatography (b), as eluting solution, uses a solution of 20 mM Tris, 1 M NaCl, pH 7.5. In one particular embodiment, the purification method of the invention further comprises a pseudo-affinity chromatography step on hydroxyapatite (c); in one preferred embodiment the separation step by pseudo-affinity chromatography is chromatography on hydroxyapatite of ceramic hydroxyapatite type.

In one preferred embodiment, the separation step by chromatography on hydroxyapatite allows the removal of high molecular weight proteins eluted with the serum amyloid P protein obtained by hydrophobic interaction chromatography at step (b). Their removal contributes towards obtaining a purified serum amyloid P component. The eluate of the serum amyloid P protein resulting from chromatography on hydroxyapatite is therefore significantly enriched. The degree of purity of the serum amyloid P protein obtained after separation step (c) by pseudo-affinity chromatography on hydroxyapatite is 94% or higher, preferably 95% or higher, more preferably 96% or higher, further preferably 97% or higher, most preferably 98% or higher.

In one particular embodiment, the purification method of the invention further comprises a virus inactivation step. Advantageously, the virus inactivation step is applied to the eluate derived from step (b). In one particular embodiment, the eluate resulting from the separation step by hydrophobic interaction chromatography (b) and virally inactivated at the virus inactivation step, is directly loaded onto the hydroxyapatite resin, the purification on hydroxyapatite allowing substantially the entire solvent and detergent to be removed that are contained in the serum amyloid P protein fraction. In one preferred embodiment, the virus inactivation step allows the inactivation of enveloped viruses making the purified amyloid P protein of the invention virally safe that is intended to be administered for diagnostic purposes, for the monitoring of treatment and/or prevention of amyloidosis. Advantageously, the virus inactivation step is conducted by solvent-detergent treatment, preferably in the presence of a Tween (polysorbate 80)—TnBP mixture, preferably a mixture with final concentration of 1% (v/v) polysorbate 80-0.3% (v/v) TnBP. In one particular embodiment, the virus inactivation treatment can also be conducted in the form of treatment with tri-n-butylphosphate, Triton X-100 or sodium cholate.

In one preferred embodiment, the separation step by chromatography on hydroxyapatite (c), as equilibration and/or wash and/or elution solution, uses a phosphate solution and preferably a sodium phosphate solution. Preferably, the separation step by chromatography on hydroxyapatite (c), as equilibration and/or wash solution, uses a phosphate solution of concentration between 5 mM and 50 mM, preferably a concentration of 10 mM. Preferably, the separation step by chromatography on hydroxyapatite (c), as equilibration and/or wash solution, uses a 10 mM Phosphate solution, pH7.5. In one particular embodiment, the separation step by chromatography on hydroxyapatite (c) comprises a first washing step with a solution of 20 mM Tris, 1 M NaCl, 0.3% TnBP, 1% Tween 80, pH7.5. Preferably separation step (c) by chromatography on hydroxyapatite, as eluting solution, uses a phosphate solution of concentration between 25 mM and 250 mM, advantageously of concentration between 50 mM and 200 mM, and preferably at a concentration of 100 mM. Preferably, the separation step by chromatography on hydroxyapatite (c), as eluting solution, uses a 100 mM Phosphate solution, pH 7.5.

The degree of purity of the serum amyloid P protein obtained after the chromatography on hydroxyapatite step (c) is 94% or higher, preferably 95% or higher, more preferably 96% or higher, further preferably 97% or higher and advantageously 98% or higher. In one particular embodiment, the purification method of the invention further comprises a separation step (d) by affinity chromatography on protein A or G, preferably affinity chromatography on protein A.

In one preferred embodiment, the affinity chromatography separation step on protein A (d), as equilibration and/or wash solution, uses a buffer solution containing a chelating agent and preferably a solution of 10 mM Tris, 10 mM EDTA, pH 8.0. In one particular embodiment, the equilibration and/or wash solution of the affinity chromatography step on protein A (d) also contains salts at a concentration of between 50 mM and 500 mM, advantageously between 100 mM and 250 mM, the salt concentration preferably being 140 mM, the salt preferably being NaCl. Advantageously, step (d) to separate by affinity chromatography on protein A, as equilibration and/or wash solution, uses a solution of 10 mM Tris, 10 mM EDTA, 140 mM NaCl, pH 8.0. The degree of purity of the serum amyloid P protein obtained after the affinity chromatography step (d) on protein A is 96% or higher, preferably 97% or higher, more preferably 98% or higher, further preferably 99% or higher. Advantageously the serum amyloid P protein obtained after the affinity chromatography separation step on protein A (d) is substantially free of contaminant proteins, and in particular free of immunoglobulins, preferably immunoglobulins M and G, and complement factor C3, this list not being limiting.

In one particular embodiment, the purification method of the invention further comprises one or more concentration and/or diafiltration steps. Preferably, after the concentration step, the concentration of eluted solution is comprised between 0.25 mg/mL and 5 mg/mL, advantageously the concentration is between 0.5 mg/mL and 2 mg/mL, and more preferably it is 1 mg/mL. In one particular embodiment, the diafiltration step is performed on a Millipore Polyethersulfone membrane (PES) 30 kDa, 50 cm².

In one particular embodiment, the diafiltration step, as wash solution, uses a solution of 10 mM Tris, 140 mM NaCl, 10 mM EDTA, pH8.0. In one particular embodiment, the diafiltration step, as wash solution, uses a solution of 10 mM Tris, 10 mM EDTA, pH 8.0. Advantageously, the eluate derived from separation step (c) by chromatography on hydroxyapatite is diafiltered prior to loading thereof onto the affinity chromatography column on protein A at step (d), against a solution of 10 mM Tris, 10 mM EDTA, pH 8.0. In one particular embodiment, the diafiltration step, as wash solution, uses a solution of 10 mM Tris, pH 8.0. Advantageously, the eluate derived from separation step (d) by affinity chromatography on protein A is diafiltered against a 10 mM Tris solution, pH 8.0.

In one particular embodiment, the purification method of the invention comprises at least one additional virus elimination step, preferably by nanofiltration, allowing the viral inactivation of the eluate of the serum amyloid P component derived from the separation step (c) by chromatography on hydroxyapatite or the separation step (d) by affinity chromatography on protein A. In another preferred embodiment of the invention, the virus removal step performed by nanofiltration is preferably conducted on filters of porosity smaller than 35 nm, preferably smaller than 20 nm, more preferably on a filter having a porosity of 15 nm, preferably on at least one Planova 15N filter (Asahi). The removal step can optionally be implemented in two steps with decreasing porosities.

In one particular embodiment, the virus removal step performed by nanofiltration is preferably conducted on Viresolve Pro Modus 1.1 filters. Advantageously, the virus removal step performed by nanofiltration on Viresolve Pro Modus 1.1 filters, as equilibration and/or wash solution, uses a solution of 10 mM Tris, 10 mM EDTA, 140 mM NaCl, pH 8.0. The nanofiltration step more particularly allows the purified amyloid P protein to be made safe against enveloped or non-enveloped viruses (viruses of poliovirus or parvovirus type) and against non-conventional transmissible agents (of prion type).

In the method of the invention, nanofiltration is preferably performed after separation step (d) by affinity chromatography on protein A since the presence of high concentrations of high molecular weight proteins in the protein extract to be filtered generally leads to clogging of the filter, all the more so when the method is implemented on an industrial scale. The purified amyloid P protein resulting from the method comprising the above-mentioned virus removal step conforms to the international recommendations issued by the EMEA or FDA regarding plasma and biotechnological products insofar as it meets the required safety conditions both for enveloped and non-enveloped viruses. In one particular embodiment, the starting material of the invention is plasma of human or non-human origin.

Advantageously, the high degree of purity of the serum amyloid P protein obtained with the method of the invention is characterized by the absence of serum contaminants such as complement factor C3, immunoglobulins (Ig), preferably immunoglobulins M and G, this list not being limiting. Advantageously, the serum amyloid P protein obtained with the method maintains its biological properties and more particularly its affinity for natural ligands of serum amyloid P protein such as phosphoethanolamine, fibronectin, the C1q molecule, collagen, phosphocholine, DNA and chromatin, immune complexes, sugars and in particular methyl 4,6-O-(1-carboxyethylidene)-β-D-galactopyranoside (MOβDG), complement components, all forms of amyloid fibrils, the C4-binding protein and glycosaminoglycans. In one particular embodiment, the serum amyloid P protein obtainable by the method of the invention is radio-labelled, advantageously with iodine 125 (125I), iodine 123 (123I), iodine 131 (131I), iodine 124 (124I), indium 111 (111In) or technetium 99m (99 mTc).

The present invention also concerns a method for determining the presence of amyloid deposits in a tissue or organ of a subject, using the purified amyloid P protein of the invention and labelled with a radio-isotope. The radio-isotopes are well known to persons skilled in the art and include for example iodine 125 (125I), iodine 123 (123I), iodine 131 (131I), iodine 124 (124I), indium 111 (111In) or technetium 99m (99 mTc), and preferably iodine 123. The purified amyloid P component radio-labelled according to the invention is administered intravenously to a subject and the subject is subsequently subjected to scintigraphic imaging. Scintigraphy is performed 24 hours after administration of the radio-labelled, purified amyloid P protein and several viewing angles are taken e.g. whole anterior, posterior views of the subject. The imaging method uses a gamma camera to detect the signal emitted by the radio-labelled, purified serum amyloid P component of the invention and thereby obtain an image of the amyloid plaque within the tissue or organ.

The present invention also concerns a method for determining amyloidosis in a subject. The radio-labelled, purified amyloid P protein of the invention is administered intravenously to a subject. Biological samples are taken 5, 15, 30, 60 minutes then 24 hours after the administering of the radio-labelled, purified amyloid P protein. Advantageously the taking of biological samples is continued for at least 48 hours after administration of the radio-labelled, purified amyloid P protein, preferably at least 72 hours, at least 6 days following after this administration. The biological samples are preferably a biological fluid, advantageously blood or urine. From these biological samples, the signal emitted by the radio-labelled purified amyloid P protein of the invention will be quantified, and a ratio is calculated between the determined quantity of protein and the quantity of administered protein. Quantification of the signal emitted by the radio-labelled, purified amyloid P protein of the invention is determined using techniques known to persons skilled in the art.

A further subject of the present invention is an antibody directed against serum amyloid P protein obtainable by the method of the invention. A further subject of the present invention is a kit for the scintigraphic determination of the presence of amyloid deposits in a tissue or organ of a subject, comprising as reagent the serum amyloid P protein obtainable by the method of the invention.

The method of the present invention is illustrated in more detailed manner by the Examples below. These Examples describe specific embodiments of the invention that cannot be construed as limiting the scope thereof.

### EXAMPLES

### Example 1

### Experimental Conditions Applied for Purification of Human Serum Amyloid P Protein

A—Starting Material

The starting material is human fresh frozen plasma. The 1^{st} step entails the overnight thawing of the pouch containing about 250 mL of human fresh frozen (about 16 to 20 hours) at a temperature of between +2° C. and +8° C. After it has thawed out, the human fresh unfrozen plasma is left at ambient temperature (25±1° C.) for 1 hour.

B—Recalcification.

Recalcification is obtained by incubating the fresh unfrozen plasma that has been left to stand at ambient temperature for 1 hour, in 10 mM CaCl₂ solution, final concentration, for 2 hours, at ambient temperature (25±1° C.) and under light agitation (100 rpm). The recalcified fresh unfrozen plasma is then centrifuged for 20 minutes at a temperature of +4° C. and speed of 4000 g. The supernatant resulting from centrifugation of the recalcified fresh unfrozen plasma is collected and its volume is about 240 mL.

C—Phenyl-Type Hydrophobic Interaction Chromatography

Phenyl-type hydrophobic interaction chromatography is conducted at ambient temperature and uses the GE Pharmacia Akta Explorer 100 chromatography system. The supernatant resulting from centrifugation of the recalcified fresh unfrozen plasma (about 240 mL) is diluted twice in a buffer equilibration solution of composition 20 mM Tris, 10 mM CaCl₂, pH 7.5. The supernatant thus diluted is passed through a column (XK16 height 15 cm, volume 30 mL) comprising a stationary phase of phenyl type (PhenylTosoh 650M) previously equilibrated with the buffer equilibration solution of composition 20 mM Tris, 10 mM CaCl₂, pH 7.5. The flow rate is preferably 7.5 mL per minute.

A 1^{st} washing of the column is carried out with a buffer wash solution of composition 20 mM Tris, 10 mM CaCl₂, pH 7.5. Two other washings of the column are successively performed with a buffer wash solution of composition 20 mM Tris, 10 mM CaCl₂, pH 7.5. Elution of the human serum amyloid P protein is then conducted by passing through the column a buffer elution solution of composition 20 mM Tris, 1 M NaCl, pH 7.5.

D—Solvent-Detergent Treatment

The eluate resulting from the separation step by phenyl-type hydrophobic interaction chromatography is subjected to virus inactivation by treatment with 1% Tween 80/0.3% TnBP, final concentration. The virus inactivation treatment is conducted for a time of 6 hours at ambient temperature (25±1° C.). The virus inactivation treatment can also be conducted in the form of treatment with tri-n-butylphosphate, Triton X-100 or sodium cholate. This step can also be conducted for a time of 16 hours at a temperature of +4° C. (4±1° C.).

E—Chromatography on Hydroxyapatite

The chromatography on hydroxyapatite step takes place at ambient temperature and uses the GE Pharmacia Akta Explorer 100 chromatography system. The eluate resulting from the separation step by phenyl-type hydrophobic interaction chromatography, and virus-inactivated, is passed through a column (C10, height 9, volume 7 mL) of type II hydroxyapatite resin, particle size 80 μm. The column is previously equilibrated with a 1^{st} equilibration solution of composition 200 mM Phosphate, pH 7.5, followed by a second equilibration solution of composition 10 mM Phosphate, pH 7.5. The flow rate is 4 mL per minute.

A 1^{st} washing of the column is performed with a buffer wash solution of composition 10 mM Phosphate, pH 7.5. A 2^{nd} washing of the column uses a buffer wash solution of composition 60 mM Phosphate, pH 7.5. Elution of the human serum amyloid P protein is then obtained by passing through the column a buffer eluting solution of composition 100 mM Phosphate, pH 7.5. The column is then regenerated with a buffer regeneration solution of composition 200 mM Phosphate, pH 7.5.

F—Concentration and Diafiltration

The eluate resulting from the chromatography on hydroxyapatite step is subjected to membrane ultrafiltration, preferably on a Polyethersulfonate (PES) membrane, 30 kDa, 50 cm² PelliconBiomax (Millipore). The concentration of the eluate resulting from the chromatography on hydroxyapatite step and ultra-filtered is 1 mg/mL. The eluate resulting from the chromatography on hydroxyapatite step, having a concentration of 1 mg/mL, is then diafiltered against 5 volumes of 10 mM Tris, 140 mM NaCl, 10 mM EDTA buffer, pH 8.0. The eluate resulting from the chromatography on hydroxyapatite step, ultra-filtered to a concentration of 1 mg/mL and diafiltered, is again concentrated on an Amicon 5 kDa membrane (Millipore) to a final volume of 5 mL.

G—Nanofiltration of the Concentrate Obtained after Chromatography on Hydroxyapatite and Ultrafiltration/Diafiltration

The filters used are filters of reference Planova 15 N (Asahi). The filters used are composed of hollow, hydrophilic cupro-ammonium cellulose fibres having a nominal pore size of 15±2 nm. The total protein yield of nanofiltration is 80%.

H—Results

H1—Analysis of Purity

The purity of the concentrate obtained after the steps of chromatography on hydroxyapatite and ultrafiltration/diafiltration, is analysed by deposit and migration on SDS-PAGE gel (NovexTris-glycine 4-20%, Invitrogen EC6025 Box) of 10 μg of the proteins of said concentrate. The following samples are also deposited on SDS-PAGE gel:
5 μL of molecular weight marker (BenchMarkproteinladder, InVitrogen, 10747-012);
20 μL of Sample Buffer (SB) solution, 1×;
BSA standards respectively corresponding to 0.2% (0.02 μg), 0.5% (0.05 μg), 1% (0.10 μg), 1.5% (0.15 μg), 2% (0.20 μg), 4% (0.40 μg) to evaluate the purity of the product.

Before being deposited on SDS-PAGE gel, each sample is heated for 5 min at +95° C. Migration of the proteins of each sample occurs under a constant voltage of 40 mA, in TGS 1× buffer.

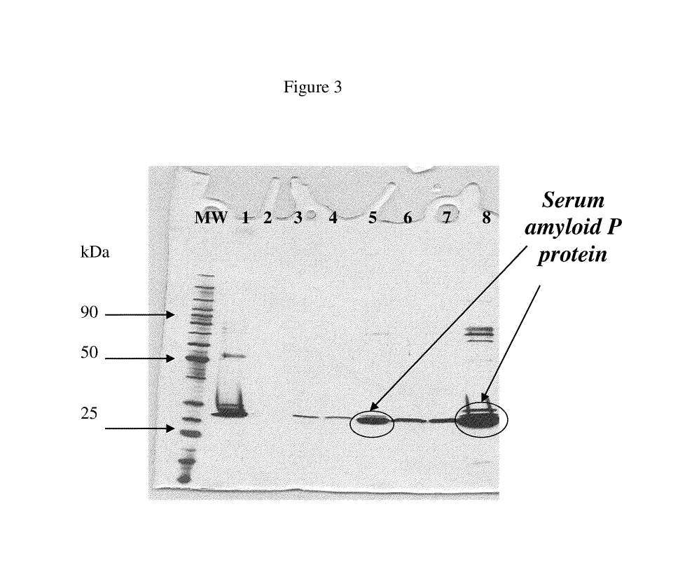

An Immunoblot is performed after electrophoresis on SDS-PAGE gel (FIG. 3). After transfer onto nitrocellulose and saturation with albumin, contact is made with a primary antibody, Rabbit anti-Serum Amyloid P protein (Calbiochem, 565191). Marking with a secondary antibody, anti-mouse AP conjugated (Promega, S373B) is carried out before detection using the NBT/BCIP technique on autoradiography film. The results are given in Table 1.

TABLE 1
Purity

| Identi- | | Volume | | | Quantification |
|---|---|---|---|---|---|
| fying | Molecular | of all | Volume of | Quan- | in % (LOQ |
| Band | weight | Bands | impurities | tification | 0.2%*) |
| 1 | 78749 | 3.75 | 3.75 | 0.13 | 0.20 |
| 2 | 70556 | 0.93 | 0.93 | 0.08 | 0.20 |
| 3 | 63711 | 0.71 | 0.71 | 0.07 | 0.20 |
| 4 | 52853 | 1.16 | 1.16 | 0.08 | 0.20 |
| 5 | 28932 | 15.21 | 15.21 | 0.34 | 0.34 |
| 6 | 27952 | 35.14 | 35.14 | 0.72 | 0.72 |
| 7 | 25504 | 2703.75 | | 0.00 | 0.00 |

| | | |
|---|---|---|
| Purity (%) | 98.6 | 98.1 |

*Limit of Quantification, values given with this limit

The IOD volume (Integrated Optical Density) of the impurities on gel is quantified by comparison with standard BSA bands. The purity of the protein is calculated by subtracting the percentage of impurities.

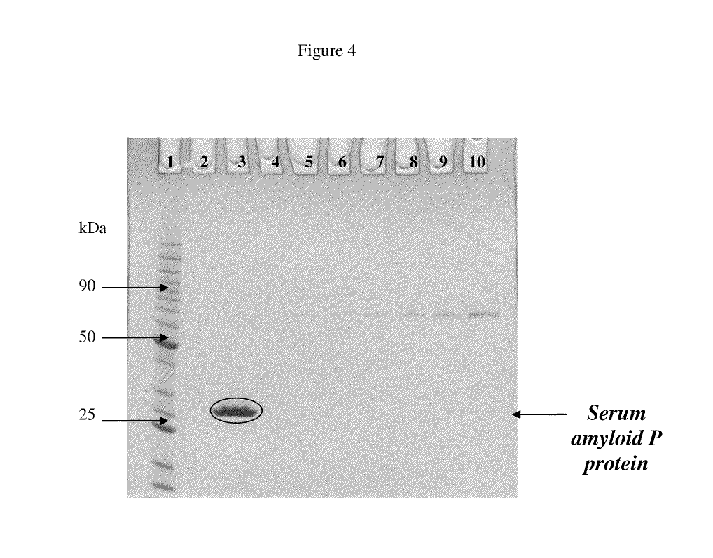

The gel is also stained with Coomassie Blue to evaluate the purity of each sample (FIG. 4). As shown in FIG. 4, the concentrate obtained after the steps of chromatography on hydroxyapatite and ultrafiltration/diafiltration (Lane 3) only exhibits one single homogeneous band. All the high molecular weight proteins have been removed from the concentrate obtained after the steps of chromatography on hydroxyapatite and ultrafiltration/diafiltration. As shown in Table 1, the purity of the concentrate obtained after the steps of chromatography on hydroxyapatite and ultrafiltration/diafiltration is 98.6%.

H2—Summary Table

The μBCA method (MicroBCAssay) was used to assay the total proteins of each sample. This is a colorimetric assay. The antigen assay of human serum amyloid P protein is conducted using the ELISA technique (Enzyme-Linked Immuno Sorbent Assay). The results are given in Table 2. The final yield before the nanofiltration step is 12 mg/L of plasma (OD 280 nm) to 17 mg/L of plasma (ELISA), i.e. 34.8 to 48.6%.

TABLE 2
Elisa/Micro BC Assay
Results of μBCA and ELISA P125

| | | μBCA | | | ELISA | | |
|---|---|---|---|---|---|---|---|
| | Volume | Concentration | | | Concentration | | |
| Sample | (mL) | (μg/mL) | mg | % | (μg/mL) | mg | % |

| | | | | | |
|---|---|---|---|---|---|
| Plasma pouch | 273 | ND | 30 | 8 | |
| n^{o}8495652 | | | | | |
| Plasma pouch | 288 | | 38 | 11 | |
| n^{o}8428627 | | | | | |
| Plasma pouch | 270 | | 26 | 7 | |
| n^{o}8428645 | | | | | |
| Plasma pouch | 293 | | 34 | 10 | |
| n^{o}8495688 | | | | | |
| Combined 4 | 1124 | | | 36 | 100 |
| pouches | | | | | |

| | | |
|---|---|---|
| Recovery | 0 | 100 |

Phenyl-type hydrophobic interaction chromatography (Toso 650M)

| | | | | | | | |
|---|---|---|---|---|---|---|---|
| Initial state | 2180 | 25615 | 55841 | 100 | 13 | 28 | 100 |
| Non-retained | 3000 | 18001 | 54003 | 7 | 0 | 0 | 0 |
| fraction | | | | | | | |
| 1^{st} washing | 750 | 166 | 125 | 0 | 0 | 0 | 0 |
| 2^{nd} washing | 750 | 44 | 33 | 0 | 0 | 0 | 0 |
| 3^{rd} washing | 1650 | 0 | 0 | 0 | 0 | 0 | 0 |
| Elution peak | 150 | 64 | 10 | 0 | 134 | 20 | 71 |
| End Elution | 600 | 0 | 0 | 0 | 1 | 1 | 2 |
| Regeneration | 600 | 738 | 443 | 1 | 0 | 0 | 0 |

| | | |
|---|---|---|
| Recovery | 98 | 73 |

Chromatography on hydroxyapatite (type II, 80 μm)

| | | | | | | | |
|---|---|---|---|---|---|---|---|
| Initial state | 160 | 299 | 47852 | 100 | 168 | 270 | 100 |
| Non-retained | 290 | 324 | 93962 | 196 | 0 | 0 | 0 |
| fraction | | | | | | | |
| 10 mM wash | 90 | 48 | 4323 | 9 | 3 | 0 | 1 |
| 60 mM wash | 90 | 0 | 0 | 0 | 0 | 0 | 0 |
| 100 mM | 35 | 386 | 13513 | 28 | 578 | 20 | 75 |
| elution | | | | | | | |
| End 100 mM | 70 | 29 | 2023 | 4 | 32 | 2 | 8 |
| elution | | | | | | | |
| Regeneration | 60 | 20 | 1221 | 3 | 17 | 1 | 4 |
| 200 mM | | | | | | | |

| | | |
|---|---|---|
| Recovered Whole Proteins | 35 | 87 |

Ultrafiltration (PES 30 kDa membrane)/Diafiltration

| | | | |
|---|---|---|---|
| Elution on | 35 | 708 | 25 |
| hydroxyapatite, | | | |
| concentrated, | | | |
| diafiltered | | | |

H3—Composition of the Serum Amyloid P Component Obtained at the Stage of Purified Bulk Raw Material (PBRM).

The composition of the SAP preparation of high purity is described in the Table below (Table 3).

TABLE 3
Formulation

| | | | |
|---|---|---|---|
| Component | | | Reference to |
| name | Formula | Function | standards |

Active ingredient

| | | | | |
|---|---|---|---|---|
| Serum amyloid | 500 | μg/mL | Amyloid marker | Internal |
| P protein | | | | Monograph |

Other components

| | | | | |
|---|---|---|---|---|
| Trisodium | 2.94 | mg/mL | Excipient | European |
| citrate | | | | Pharmacopeia |
| Sodium | 8.76 | mg/mL | Excipient | European |
| chloride | | | | Pharmacopeia |
| Proteins | 520-540 | μg/mL | Serum amyloid | — |
| | | | P protein and | |
| | | | residual allogeneic | |
| | | | proteins | |

The excipients trisodium citrate and sodium chloride are added in buffer form and via their functional properties ensure: the final pH of the preparation (physiological), the osmolality of the preparation (isotonic). Conformity with the manufacturing formula is determined by controls carried out on the PBRM (Elisa SAP, Protein level, Osmolality, pH, endotoxins, Bacterial count, SAP activity and functionality tests).

Validation of Purification.

Analysis of the results on the three batches examined shows good reproducibility of the method for manufacturing the concentrate of purified amyloid P protein at industrial level, and a considerable improvement in yield (12 to 17 mg/L of plasma i.e. 34.8 to 48.6%), together with the obtaining of a concentrate of SAP protein having a high degree of purity.
Analysis of the fractions before nanofiltration, and PBRM before and after bacteriological filtration on the three examined batches shows: the efficacy of nanofiltration and bacteriological filtration in the three batches examined. The microbial innocuousness of the amyloid P protein preparations is demonstrated.
TNBP and Tween 80: the residual concentrations of Tween 80 and TNBP are lower than the standard set by the European Pharmacopeia for the three batches examined.
Impurities of microbial origin: the microbial innocuousness of the amyloid P protein preparations is demonstrated and conforms to specifications.
Electrophoretic purity (SDS-PAGE gel): the electrophoretic plotting identified in Lane 3 evidences a band of about 25 kDa recognized by the anti-serum amyloid P protein antibody corresponding to the purified amyloid P component. No allogeneic contaminant was evidenced.

### Example 2

### Evidencing of the Reproducibility of the Method of the Invention

I. Purification Scheme

The applied purification scheme is the following:

A—Starting Material

The starting material is fresh frozen human plasma. The 1^{st} step consists of the thawing of 35 pouches of fresh frozen human plasma (about 300 ml per pouch) at ambient temperature overnight. The unfrozen pouches are combined in a biotainer to form a pool of fresh unfrozen plasma, transferred to a 20-litre pouch then incubated under agitation (Wave mixer) until the temperature of the pool of fresh unfrozen plasma reaches 20° C.

B—Recalcification

Recalcification is carried out by adding to the pool of fresh unfrozen human plasma a solution of 1M CaCl₂ to obtain a final concentration of 10 mM, followed by incubation for 2 hours at ambient temperature. The pool of recalcified fresh unfrozen plasma is then centrifuged at a speed of 4000 g for 20 minutes at ambient temperature. The supernatant resulting from centrifugation of the pool of recalcified fresh unfrozen plasma is harvested.

C—Phenyl-Type Hydrophobic Interaction Chromatography

The supernatant resulting from centrifugation of the pool of recalcified fresh unfrozen plasma is diluted twice in a buffer equilibration solution of composition 20 mM Tris, 10 mM CaCl₂, pH 7.5. The supernatant thus diluted is passed through a column (BPG 140 height: 20 cm, volume: 3078 ml) comprising a stationary phase of phenyl type (PhenylTosoh 650M) previously equilibrated with the buffer equilibration solution of composition 20 mM Tris, 10 mM CaCl₂, pH 7.5, at a flow rate of 400 ml/min.

A first washing of the column is carried out with a buffer wash solution of composition 20 mM Tris, 10 mM CaCl₂, pH 7.5. Three other washings of the column are successively performed with 20 mM Tris, 10 mM CaCl₂, 150 mM NaCl solution, pH 7.5; 20 mM Tris, 10 mM CaCl₂, 500 mM NaCl solution, pH 7.5; 20 mM Tris, 10 mM CaCl₂, 1 M NaCl solution, pH 7.5. Elution of the human serum amyloid P protein is then performed by passing through the column a buffer eluting solution of composition 20 mM Tris, 1 mM NaCl, pH 7.5.

The steps of thawing fresh frozen human plasma, recalcifying the fresh unfrozen human plasma and separation by phenyl-type hydrophobic interaction chromatography are carried out twice so as to treat the equivalent of 19 litres to 20 litres of plasma. The two eluates derived from the separation step by phenyl-type hydrophobic interaction chromatography are combined in a pool in a 20-litre pouch before proceeding with the virus inactivation step by solvent-detergent treatment.

D—Virus Inactivation Step by Solvent-Detergent Treatment

The pool of eluates, resulting from the separation step by phenyl-type hydrophobic interaction chromatography, is subjected to virus inactivation by solvent-detergent treatment. Solvent-detergent treatment takes placed by adding to the pool 3% TnBP, 10% Tween80 to obtain a final concentration of 0.3% TnBP and 1% Tween80. The mixture thus obtained is brought to a temperature of 25° C. and incubated for 6 hours at 25° C. under agitation.

E—Separation Step by Affinity Chromatography on Hydroxyapatite

The pool of eluates resulting from the separation step by phenyl-type hydrophobic interaction chromatography, and virally inactivated, is passed through a column (Millipore Vantage 60 height: 15 cm; volume: 424.5 ml) of type II hydroxyapatite resin, particle size 80 μm, at a flow rate of 160 ml/min. The column is previously equilibrated with a first equilibration solution of composition 200 mM Na Phosphate, pH 7.5, followed by a second equilibration solution of composition 10 mM Na Phosphate, pH 7.5.

A first washing of the column is carried out with a buffer wash solution of composition 20 mM Tris, 1 M NaCl, 0.3% TnBP, 1% Tween 80, pH 7.5. A second washing of the column is carried out with a buffer wash solution of composition 10 mM Na Phosphate, pH 7.5. The column is washed a third time with a buffer wash solution of composition 60 mM Na Phosphate, pH 7.5. Elution of the human serum amyloid P protein is then conducted by passing through the column a buffer eluting solution of composition 100 mM Na Phosphate, pH 7.5.

F—Concentration and Diafiltration Step by Tangential Ultrafiltration (UFT)

The eluate resulting from the chromatography step on hydroxyapatite is subjected to a concentration and diafiltration step on membrane of PES type (Millipore Pellicon 2, cut off: 30 kDa, surface: 0.1 m²). The membrane is previously equilibrated with an equilibration solution of composition 10 mM Tris, 140 mM NaCl, 10 mM EDTA, pH 8.

The eluate resulting from the chromatography step on hydroxyapatite is concentrated to a final volume of 300 ml (TMP=1.4 bars), then diafiltered against 5 volumes of 10 mM Tris, 140 mM NaCl, 10 mM EDTA buffer, pH 8. The eluate resulting from the chromatography step on hydroxyapatite, and diafiltered, is again concentrated to a volume of 250 ml. The membrane is then rinsed with 250 ml of 10 mM Tris, 140 mM NaCl, 10 mM EDTA buffer solution, pH 8, and the rinsing solution and the eluate resulting from the chromatography step on hydroxyapatite are diafiltered and concentrated and then combined (final volume of the retentate about 500 ml).

G—Separation Step by Affinity Chromatography on Protein A

The separation step by chromatography on protein A uses a column (Vantage 44 height: 10 cm; volume: 150 ml) comprising Mab Select Sure resin previously equilibrated with a buffer equilibration solution of composition 10 mM Tris, 10 mM, EDTA, 140 mM NaCl, pH 8.0, at a flow rate of 8 ml/min. After loading the retentate, the column is washed with a wash solution of composition 10 mM Tris, 10 mM EDTA, 140 mM NaCl, pH 8.0. The outgoing flow is collected and filtered on 0.22 micron filters.

H—Nanofiltration Step

The nanofiltration step is performed on filters of reference Viresolve Pro Modus 1.1 (Millipore, surface: 170 cm²) previously equilibrated with an equilibration solution of composition 10 mM Tris, 10 mM EDTA, 140 mM NaCl, pH 8.0. The flow resulting from the separation step by affinity chromatography on protein A is filtered and the filters are rinsed with a wash solution of composition 10 mM Tris, 10 mM EDTA, 140 mM NaCl, pH 8.0. The total protein yield of nanofiltration is 100%.

I—Concentration and Diafiltration Step The filtrate resulting from the nanofiltration step is subjected to a step for concentration and membrane diafiltration, preferably a PES membrane (Pellicon 2, cut off: 30 kDa, surface: 0.1 m²) previously equilibrated with an equilibration solution of composition 10 mM Tris, pH 8. The filtrate resulting from the nanofiltration step is concentrated to a volume of 200 ml (TMP=1.4 bars), then diafiltered against 5 volumes of 10 mM Tris, pH 8. The filtrate resulting from the nanofiltration step and diafiltered is again concentrated to a volume of 150 ml. The filters are then rinsed with 100 ml of 10 mM Tris solution, preferably at pH 8. The outgoing flow and the filtrate, resulting from the nanofiltration step, diafiltered and concentrated, are combined then filtered on 0.22 μm.

II. Results

A—Analysis of the Reproducibility of the Three Batches after the Separation Step by Chromatography on Hydroxyapatite (HA).

To evaluate the reproducibility of the developed manufacturing method, 3 reproducibility batches were prepared on an industrial scale paying identical heed to the purification scheme.

A1—Analysis of the Yield Obtained with the First Reproducibility Batch.

Elisa assay allows an estimation to be given of the quantity of SAP present in the starting plasma pool, and after recalcification. The variability thereof does not however allow an estimation of the yields at the later steps of the method. For these steps, the concentrations are estimated using the BCA assay or Bradford method (UFT last step). The global yield can be estimated by compiling the ELISA and Bradford data.

TABLE 4
Yields
Results of Protein Content Analyses (BCA), Bradford (UFT step) and ELISA P125

| | Vol. | BCA | | | Elisa | | |
|---|---|---|---|---|---|---|---|
| Sample | (ml) | cc μg/ml | mg | % | cc μg/ml | mg | % |

PhenylTosoh #1 650M

| | | | | | | | |
|---|---|---|---|---|---|---|---|
| Start | 17782 | 24365 | 433258 | 100% | 24 | 427 | 100% |
| FT | 26080 | 16936 | 441691 | 102% | 1 | 26 | 6% |
| Wash 1 | 6000 | 138 | 828 | 0.19% | 0 | 0 | 0% |
| Wash 2 | 6000 | 31,89 | 191 | 0.04% | 0 | 0 | 0% |
| Wash 3 | 12000 | 0 | 0 | 0.00% | 0 | 0 | 0% |
| Elution | 2500 | 54 | 135 | 0.03% | 113 | 283 | 66% |
| Regeneration | 5000 | 472 | 2361 | 0.54% | 4 | 20 | 5% |

| | | |
|---|---|---|
| Yield | 103% | 77% |

PhenylTosoh #2 650M

| | | | | | | | |
|---|---|---|---|---|---|---|---|
| Start | 17400 | 21036 | 366026 | 100% | 26 | 45 | 100% |
| FT | 25300 | 10920 | 276276 | 75% | 0 | 0 | 0% |
| Wash 1 | 6000 | 89 | 531 | 0.15% | 0 | 0 | 0% |
| Wash 2 | 6300 | 32 | 202 | 0.06% | 0 | 0 | 0% |
| Wash 3 | 12600 | 12 | 152 | 0.04% | 0 | 0 | 0% |
| Elution | 3400 | 42 | 141 | 0.04% | 85 | 289 | 64% |
| Regeneration | 6000 | 361 | 2168 | 1% | 2 | 12 | 3% |

| | | |
|---|---|---|
| Recovery | 76% | 67% |

Hydroxyapatite type II 80 μm

| Start | 6574 | Non-assayable | | | 68 | 449 | 100% |
|---|---|---|---|---|---|---|---|
| FT | 8313 | | | | 0 | 0 | 0% |
| Wash 10 mM | 1250 | | | | 18 | 23 | 5% |
| Wash 60 mM | 1240 | 14 | 17 | 6% | 0.47 | 1 | 0% |
| Elution 100 mM | 1120 | 250 | 280 | 101% | 183 | 205 | 46% |
| Regeneration | 840 | 77 | 65 | 23% | 60 | 50 | 11% |
| 200 mM | | | | | | | |
| Total protein yield | | | | 131% | | | 62% |

UFT Pellicon 2 PES 0.1 m²

| | BRADFORD | | | | Elisa | | |
|---|---|---|---|---|---|---|---|
| | Vol. ml | cc μg/ml | mg | % | cc μg/ml | mg | % |
| Start | 1120 | 271 | 304 | 100% | 183 | 1208 | 100% |
| Permeate | 3200 | Non-assayable | | | 79 | 253 | 21% |
| Retentate | 510 | 549 | 280 | 92% | 1697 | 865 | 72% |
| Filtered retentate | 510 | 544 | 277 | 91% | 1360 | 694 | 57% |

Interference of Tween/TnBP with BCA and Bradford assays
Interference of EDTA with BCA assay, but compatible with Bradford

With the Bradford method, the final quantity of SAP is ±277 mg for 19 I of plasma used (64 pouches) i.e. a productivity of 14.6 mg of serum amyloid P protein per litre of plasma.

A2. Analysis of the Yields Obtained with the Three Reproducibility Batches.

If reference is made to a mean SAP concentration of 20 μg/ml in the plasma (concentration measured by ELISA throughout development), and taking into account Bradford elution data, it is possible to infer the yields per step described in the following Table (repro meaning reproducibility batch).

TABLE 5
Yields
Assay with the Bradford method

| | Repro 1 | | Repro 2 | | Repro 3 | |
|---|---|---|---|---|---|---|
| | Protein | | Protein | Yield | Protein | |
| | content | Yield | content | of | content | Yield of |
| Step | (mg) | of step | (mg) | step | (mg) | step |
| Start Phenyl | 720 | | 720 | | 720 | |
| Start HA | 449 | 62% | 422 | 59% | 408 | 57% |
| Elution HA | 280 | 62% | 240 | 57% | 240 | 59% |
| Purified bulk | 277 | 100% | 288 | 120% | 224 | 93% |

| | | | |
|---|---|---|---|
| Global yield | 38% | 40% | 31% |

| | |
|---|---|
| Mean global yield | 36.33% |

| | | | |
|---|---|---|---|
| Productivity (mg | 14.6 | 15.2 | 11.8 |
| SAP/litre of | | | |
| plasma) | | | |

| | |
|---|---|
| Mean productivity (mg | 13.87 |
| SAP/litre of plasma) | |

B—Analysis of the Purity of the Human Serum Amyloid P Protein Obtained with the Three Reproducibility Batches.

B1—Analysis of the Impurities after the Separation Step by Affinity Chromatography on Hydroxyapatite.

Method of Analysis

10 μg of UFT-filtered retentate (purified bulk) were deposited on SDS-PAGE gel under reducing and non-reducing conditions. After staining with Coomassie Blue, the impurity bands were extracted. Each sample was subjected to previous carboxylation with iodoacetamide before overnight treatment with trypsin at 37° C.

The trypsic peptides were then automatically analysed by LCMSMS. Analyses were processed using the WARPLC (Bruker) computer system. Sequence comparisons were performed with NCBI and SwissProt databases using the MASCOT operating system. Only those proteins having non-negligible identification probability were retained.

Results

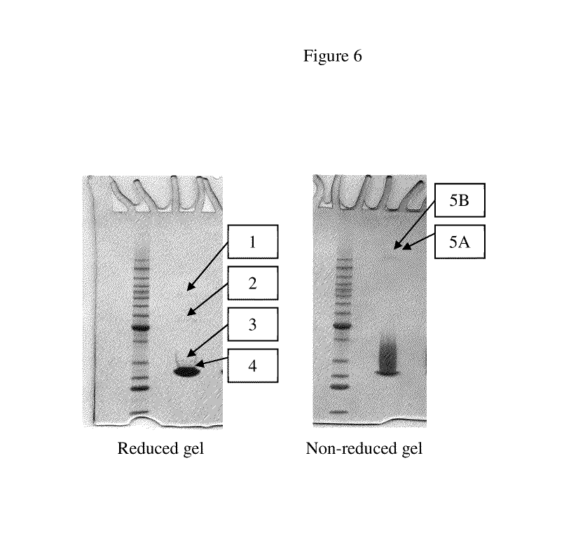

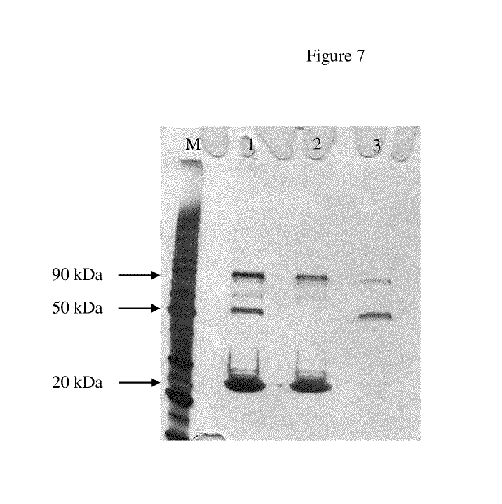

Five major impurities were characterized and their composition is given in Table 6 below, these impurities being present on the gel in FIG. 6.

TABLE 6
Impurities

| Band N^{o} | Composition (decreasing abundance) |
|---|---|
| 1 | IG mu chain C region |
| | SAP |
| | Igheavychain V-III region BRAU |
| | Igheavychain V-III region CAM |
| 2 | Ig gamma 1 Chain C |
| | SAP |
| | Ig gamma 2 Chain C |
| | Ig heavy chain V-I |
| | Igmu heavy chain |
| 3 | SAP |
| | Ig gamma 1 chain C |
| 4 | SAP |
| | Iglamda chain |
| 5A | SAP |
| | Complement factor C3 |
| | Ig gamma 1 chain |
| | Ig Kappa chain |
| 5B | SAP |
| | Ig gamma 1 chain C |
| | Ig Kappa 1 chain C |
| | Ig gamma 3 chain C |
| | Ig gamma 2 chain C |
| | Ig Kappa chain V-III |
| | Ig Kappa Chain V-IA |

Analysis of the bands shows that all the impurities are formed of SAP (or a fragment thereof) bound to another protein. The most frequently found contaminant is derived from immunoglobulins, chiefly IgG and IgM. SAP is largely in majority except in bands 1 and 2 corresponding to impurities of 50 and 80 kDa. Purity was determined in conservative manner but some impurities observed could be artefacts related to the denaturing conditions (presence of SDS) at the time of SDS-PAGE assay, which would increase the degree of purity of the solutions.

B2—Analysis of Impurities after the Separation Step by Affinity Chromatography on Protein a (Mab Select Sure).

Analysis of the purity of the human serum amyloid P protein was performed by the depositing on gel of 10 μg of UFT-filtered retentate (Purified bulk). BSA standards respectively corresponding to 0.2%, 0.5%, 1%, 1.5%, 2% and 4% of 10 μg were also deposited to evaluate the purity of the product by spectrodensitometry.

Results

The following Table summarises the purity results obtained with the three batches, under reducing or non-reducing conditions applied for SDS-PAGE gels.

TABLE 7
Purities

| | Purity after | | | |
|---|---|---|---|---|
| | separation step by | | Purity after separation | |
| | chromatography on | | step by chromatography | |
| | hydroxyapatite | | on Protein A | |
| | reducing | non-reducing | reducing | non-reducing |
| | condition | condition | condition | condition |
| Reproducibility | 95.20% | 95.20% | 96.70% | 97.70% |
| Batch 1 | | | | |
| Reproducibility | 95.00% | 95.40% | 96.70% | 97.30% |
| Batch 2 | | | | |
| Reproducibility | 93.20% | 95.10% | 96.80% | 97.60% |
| Batch 3 | | | | |
| Mean | 94.5% | 95.2% | 96.7% | 97.5% |

C—Analysis of the Activity of the Human Serum Amyloid P Protein Obtained with the Method of the Invention.

The activity of the human serum amyloid P protein obtained with the method of the invention was evidenced by measuring its binding capacity with known ligands of this protein such as fibronectin.

C1—Ligand Binding Assay

The binding of the human serum amyloid P protein obtained with the method of the invention was measured using the protocol described by Bottazzi B et al, 1997 (Bottazzi et al, “Multimer formation and ligand recognition by the long pentraxin PTX3”, J. BiolChem. 1997, vol. 272, 52: 32817-32823). The experiments were conducted at 37° C. A coating substrate was coated with the C1q molecule, type IV collagen, fibronectin or gelatin, at constant or variable concentration (increasing concentration), in the presence of PBS 1× buffer solution free of calcium ions.

The human serum amyloid P protein obtained with the method of the invention was diluted in PBS 1×, 0.05% Tween 20, milk (Régilait, saturating agent) buffer solution comprising calcium ions, then deposited in constant or variable concentration (increasing concentration) on the coated support. After incubation and washing, the coated support was incubated with a mouse monoclonal primary antibody directed against the serum amyloid P protein (clone 5.4A), washed again and then incubated with a secondary antibody allowing detection with a system of Streptavidin peroxidase/TMB type. The absorption values were read at 450-620 nm. The binding assay, as positive control, used a human serum amyloid P protein that is commercially available (standard).

C2—Results

Binding Inhibition Assay

An inhibition assay of the binding of the serum amyloid P protein obtained with the method of the invention to the coated ligand (in this case the C1q molecule or collagen) was carried out using the CRP protein (C-reactive protein) as inhibitor, which is known to be capable of binding with such coating ligands. The results show an inhibitory effect of the CRP protein on the binding of the serum amyloid P protein obtained with the method of the invention (data not presented).

Competitive Binding Assay

A competitive binding assay was carried out by measuring the optical density values after contacting the coated substrate bound to a ligand of constant concentration with a solution comprising a constant concentration of human serum amyloid P protein obtained with the method of the invention or commercially available serum amyloid P protein (standard protein), and at an increasing concentration of coating ligand.

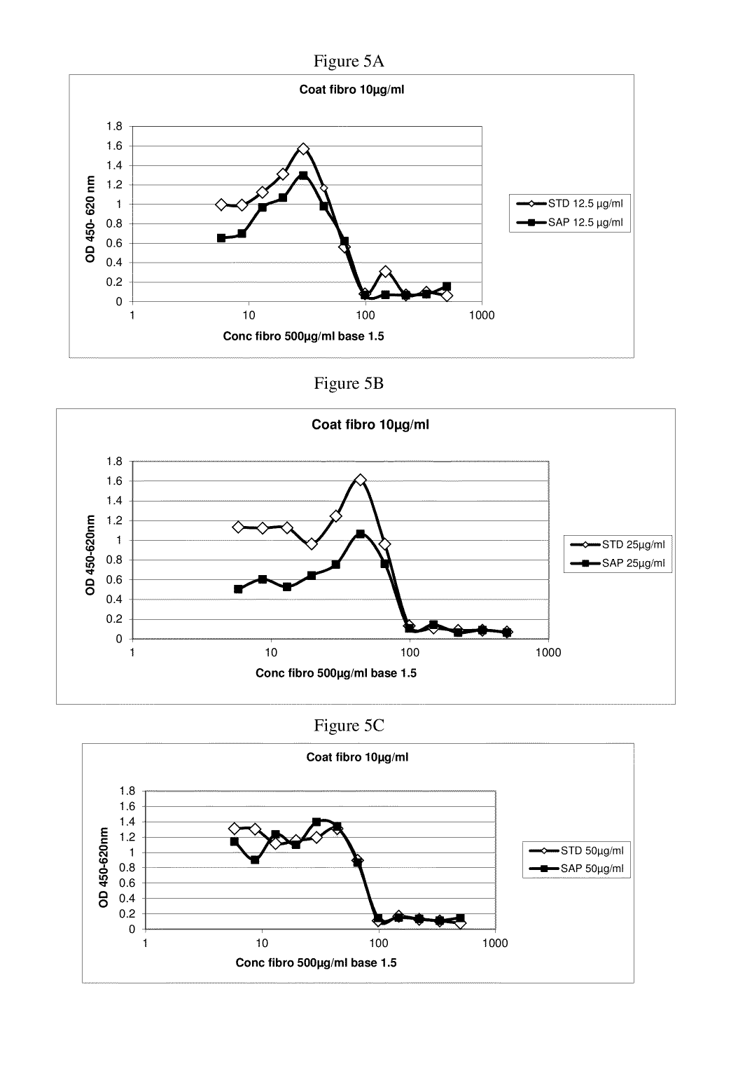

FIG. 5 shows a curve of the results obtained using fibronectin as coating ligand fixed to the substrate at a concentration of 10 μg/ml, and for concentrations of human serum amyloid P protein obtained with the method of the invention or commercially available serum amyloid P protein of 12.5 μg/ml (FIG. 5A), 25 μg/ml (FIG. 5B) and 50 μg/ml (FIG. 5C). The results in FIG. 5 show that at the three tested concentrations of serum amyloid P protein obtained with the method of the invention and of the standard protein, the plotted curves are of sigmoid shape. The results in FIG. 5 also show that at the three tested concentrations of the serum amyloid P protein obtained with the method of the invention and of the standard protein, the shape of the curve obtained with the serum amyloid P protein obtained with the method of the invention overlays the curve obtained with the standard protein. The results thus obtained show that the serum amyloid P protein obtained with the method of the invention preserves its binding capacity with fibronectin.

1. A method for purifying serum amyloid P protein from a starting material, the method comprising the following steps:
a) a recalcification step; and
b) a separation step by hydrophobic interaction chromatography.

2. The purification method according to claim 1, wherein the recalcification step (a) corresponds to incubation of the biological sample in CaCl₂ solution, at a final concentration of between 1 mM and 50 mM.

3. The purification method according to claim 1, further comprising implementing the separation step by hydrophobic interaction chromatography (b) on a stationary phase comprising phenyl-type hydrophobic groups.

4. The purification method according to claim 1, further comprising separating by pseudo-affinity chromatography on hydroxyapatite.

5. The purification method according to claim 1, further comprising separating by affinity chromatography on protein A or protein G.

6. The purification method according to claim 1, further comprising a virus inactivation step.

7. The purification method according to claim 1, further comprising at least one of: an ultrafiltration and diafiltration step.

8. The purification method according to claim 1, further comprising at least one additional virus removal step performed by nanofiltration.

9. The purification method according to claim 1, wherein the starting material is plasma or serum of human or non-human origin.

10. An amyloid P protein, obtainable by the method according to claim 1.

11. The amyloid P protein according to claim 10, wherein the binding capacity of the protein with a natural ligand of serum amyloid P protein is preserved after the method.

12. The amyloid P protein according to claim 10, wherein or it has strong affinity for amyloid fibrils and binds to the binding site of amyloid fibrils.

13. The amyloid P protein according to claim 10, further comprising radiolabelling the protein with one of: iodine 125 (125I), iodine 123 (123I), iodine 131 (131I), iodine 124 (124I), indium 111 (111In) or technetium 99m. (99 mTc).

14. A method for determining the presence of amyloid deposits in a tissue or organ of a subject, the method comprising:
i. systemic administering of an amyloid P protein by radiolabelling the protein with at least one of: iodine 125 (125I), iodine 123 (123I), iodine 131 (131I), iodine 124 (124I), indium 111 (111In) or technetium 99m. (99 mTc);
ii. using a gamma camera, detecting a signal in the tissue or organ, the signal being emitted by the protein; and
iii. obtaining at least one image of the tissue or organ, the image being obtained as output data from a gamma camera.

15. A method for determining the presence of amyloid deposits in a tissue or organ in a subject, the method comprising:
i. systemic administering of an amyloid P protein by radiolabelling the protein with at least one of: iodine 125 (125I), iodine 123 (123I), iodine 131 (131I), iodine 124 (124I), indium 111 (111In) or technetium 99m. (99 mTc);
ii. quantifying, in a biological sample of the subject, the signal emitted by the administered protein; and
iii. determining a ratio between the quantity of protein quantified at step (ii) and the quantity of protein administered at step (i).

16. The determination method according to claim 15, wherein the biological sample is a biological fluid, preferably blood or urine.

17. The determination method according to claim 15, further comprising determining the quantification of the signal emitted at step (ii) at least 24 hours after administration of the protein at step (i).

18. The amyloid P protein according to claim 10, further comprising at least one of: diagnosing or monitoring of treatment, or the therapeutic and/or prophylactic treatment of amyloidosis including Alzheimer's disease, in a human subject.

19. The amyloid P protein according to claim 10, further comprising at least one of: diagnosing or treating of type II diabetes in a human subject.

20. The amyloid P protein according to claim 10, further comprising at least one of: diagnosing or treating of autoimmune diseases in a human subject.

21. An antibody directed against the amyloid P protein according to claim 9.

22. A kit for quantifying by scintigraphy the presence of amyloid deposits in a tissue or organ of a subject, comprising as reagent the amyloid P protein according to claim 12.
